(19) United States
(12) Patent Application Publication
(10) Pub. No.: US 2006/0058351 A1
(43) Pub. Date: Mar. 16, 2006

(54) Novel ligands that modulate LXR-type receptors and cosmetic/pharmaceutical applications thereof

(75) Inventors: Philippe Diaz, Nice (FR); Jean-Michel Bernardon, Nice (FR); Etienne Thoreau, Saint Vallier De Thiey (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA RESEARCH & DEVELOPMENT, S.N.C., VALBONNE SOPHIA ANTIPOLIS (FR)

(21) Appl. No.: 11/212,714

(22) Filed: Aug. 29, 2005

Related U.S. Application Data
(63) Continuation of application No. PCT/EP04/02396, filed on Feb. 19, 2004.
(60) Provisional application No. 60/454,345, filed on Mar. 14, 2003.

(30) Foreign Application Priority Data
Feb. 28, 2003 (FR) 03/02478

(51) Int. Cl. A61K 31/454; C07D 211/32; A61K 31/445; C07D 403/02

(52) U.S. Cl. 514/317; 514/326; 546/210; 546/225

(57) ABSTRACT

Novel compounds that are ligands of the LXR receptors have the following structural formula (I): 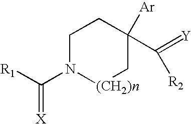 and are formulated into pharmaceutical compositions useful in human or veterinary medicine, or alternatively into cosmetic compositions.

### CROSS-REFERENCE TO PRIORITY/PCT/PROVISIONAL APPLICATIONS

This application claims priority under 35 U.S.C. § 119 of FR 03/02478, filed Feb. 28, 2003, and of provisional application Ser. No. 60/454,345, filed Mar. 14, 2003, and is a continuation of PCT/EP 2004/002396, filed Feb. 19, 2004 and designating the United States (published in the English language on Sep. 10, 2004 as WO 2004/076418 A1), each hereby expressly incorporated by reference and each assigned to the assignee hereof.

### BACKGROUND OF THE INVENTION

1. Technical Field of the Invention

The present invention relates to novel compounds that are ligands and modulators of LXR receptors, to a process for preparing them and to the formulation of at least one selective ligand of LXR-type receptors into pharmaceutical or cosmetic compositions, these compositions being useful in a regime or regimen to treat disorders, complaints or afflictions associated with the LXR receptors.

2. Description of Background and/or Related and/or Prior Art

The LXR receptors (liver X receptors) belong to the superfamily of steroidal/thyroid receptors. In 1995, P. Willy and J. Mangelsdorf cloned a novel receptor belonging to the superfamily of steroidal/thyroid receptors, referred to as LXRα (liver X receptor), by low-stringency screening of a library of complementary DNA from human liver with a pool of degenerate oligonucleotides corresponding to the DNA binding domain of the RARα nuclear receptors. Comparison of the nucleotide sequence of human LXRα with other receptors already known showed strong similarities between two sequences of orphan receptors: 77% homology with the human receptor NER-1 or Ubiquitous Receptor UR, consequently described as the second LXR subtype and referred to as LXRβ, and 92% homology with the rat receptor RLD-1, which appears to be the murine homologue of hLXRα. The LXRβ isoform shows very great homology with an orphan receptor cloned in 1993 in rats: OR-1.

Analysis by in situ hybridization and northern blot experiments of the messenger RNAs of the two human LXR subtypes identified and described: LXRα and LXRβ, demonstrates increased tissue distribution in organs with intense metabolic activity, for instance the kidneys, the liver, the intestines and, to a lesser extent, in the spleen, the adrenal glands and the skin. The hLXRβ isoform is much more ubiquitous and is also present in the brain, the testicles and the ovaries. These receptors have the capacity of forming functional heterodimers with the retinoid X receptors (RXRs). In the form of a heterodimer with the retinoid X receptors (known as the RXRs), the LXR receptors activate transcription by binding to specific DNA sequence elements, known as the response elements (LXRE), located in the promoter of the target gene whose transcription they regulate.

At the present time, only one LXRE binding site is known, characterized in the promoter of the CYP7α gene of rat (cholesterol 7-α-hydroxylase), which codes for an enzyme involved in a key step of conversion of cholesterol into bile acids and is strongly expressed in the liver.

The identification of specific LXR ligands was performed by Janowsky et al. They thus showed that only one specific oxysterol group having a cholesterol skeleton and structure was capable of activating the LXR receptors. Study of the structure/activity relationships revealed the engagement of a 3β-hydroxy group of cholesterol and an additional hydroxyl group preferably located on a side chain of the molecule. These compounds were shown to be active at their physiological concentration and more particularly a compound synthesized by the body: 22(R)-hydroxycholesterol, which is described as the most powerful activator.

A controlled proteolytic digestion experiment established that this compound is a potential LXRα ligand.

LXRα receptor activators have been described in WO 98/32444. These compounds are especially: 7α-hydroxycholesterol, 27-hydroxycholesterol, 4β-hydroxycholesterol, 24-hydroxycholesterol, 20(S)-hydroxycholesterol, 22(R)-hydroxycholesterol and 20,22-dihydroxycholesterol, have a therapeutic application in the restoration of the skin's barrier function, the induction of differentiation and the inhibition of proliferation.

Certain of these compounds, produced by the action of P450 cytochromes, are intermediates leading to bile acids or to steroid hormones, but most result from an auto-oxidation of free cholesterol or of its esters. These degradation products then participate in the system of repression of cholesterol synthesis. Despite the knowledge of this system, all of the mechanisms involved in cholesterol homeostasis have not been elucidated.

The tissue distribution of the LXRα messenger RNAs revealed a strong preponderance of these messengers in organs with metabolic activity, for instance the liver, the kidneys and the intestines, and also presence to a lesser extent in the spleen, the adrenal glands and the skin. In parallel, the tissue distribution of the LXRβs was shown to be more ubiquitous, especially with presence in the brain and the testicles.

More recently, it has been described in WO 98/32444 that FXR, PPARγ and LXRβ receptor activators are capable of restoring the barrier-function role. These activators are also presented as increasing differentiation by inhibiting epidermal proliferation.

Specifically, the skin has a structure that gives it numerous properties and a major role in the barrier function. This regulation of the barrier function is particularly provided by the epidermis.

Natural human epidermis is mainly composed of three types of cell, namely the keratinocytes, which are in the vast majority, the melanocytes and the Langerhans cells. Each of these cell types contributes via its intrinsic functions towards the essential role played in the body by the skin.

The epidermis is continually being formed by proliferation of the basal cells of the epidermis. The keratinocytes formed in the deepest part of the epidermis migrate towards the surface of the skin. During this migration, the keratinocytes differentiate by means of profound biochemical and structural changes to result in the formation of cells lacking their nucleus and their cytoplasmic organelles, but which have synthesized a horny envelope: these are the corneocytes. The horny envelope gives the corneocytes great rigidity and provides the stratum corneum with mechanical strength. The corneocytes together constitute the horny layer or stratum corneum, the outermost layer of the epidermis and main regulator of the skin's barrier function.

The cells constituting the epidermis are delimited by a lipid domain. The epidermal lipids are mainly synthesized in the live epidermis. They consist essentially of phospholipids, sphingolipids, cholesterols, free fatty acids, triglycerides, cholesterol esters and alkanes. During cell differentiation, the phospholipids, whose role consists in developing the fluid structure of the cell membranes of the live layers of the epidermis, are gradually replaced with a mixture predominantly composed of fatty acids, cholesterol and sphingolipids, which are essential constituents of the horny layer of the epidermis (stratum corneum).

In this respect, the intercellular level of cholesterol was described by Schmidt et al., The Journal of Investigative Dermatology, No. 5, 771-775; as a predominant factor in the spontaneous formation of the horny envelope.

It is observed, for example, that there is an increase in the level of phosphorylation and the level of messenger RNA of the enzymes associated with de novo synthesis of the three key types of lipids of cell maturation: serine palmitoyl transferase for the formation of ceramides, HMGCoA reductase involved in the synthesis of cholesterol and its derivatives, and acetyl CoA carboxylase and fatty acid synthases involved in the formation of the cutaneous fatty acids. It appears that the capacity to modify cell maturation, and more particularly to restore an effective barrier function, is directly linked to regulation of the synthesis of the key lipids.

Deregulation of the barrier function, whether generalized or localized, is known to be an important component of many disorders and diseases of the skin and mucous membranes. This disruption of the barrier function can result in the entry of pathogens across the affected part of the skin, but is also found to be a factor aggravating numerous skin pathologies correlated with disorders of differentiation and/or proliferation of epidermal cells.

To treat these imbalances in barrier function, and also skin disorders associated with insufficient epidermal differentiation and/or excessive proliferation of the epidermal cells, different pharmaceutical approaches have been attempted.

Considerable research is currently being conducted into finding compounds that can regulate the function of the horny layer, and also develop an action on epidermal differentiation and proliferation. However, no treatment at the present time is entirely satisfactory, especially on account of the side effects induced by the known compounds. Thus, there is a serious need to improve the existing treatments by investigating novel derivatives that are more active and that can be used while limiting the adverse side effects.

### SUMMARY OF THE INVENTION

The present invention thus features novel compounds that are ligands of the LXR receptors, having the following structural formula (I): 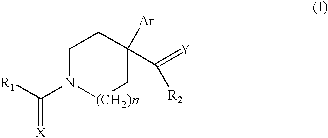
in which:
R₁ is:
   (i) an alkyl radical having from 1 to 12 carbon atoms or an aryl, aralkyl, aralkenyl or heteroaryl radical,
   (ii) a radical: 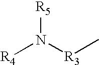
      wherein R₃, R₄ and R₅ are as defined below,
   (iii) a radical: 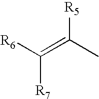
      wherein R₆ and R₇ are as defined below,
   (iv) a radical: 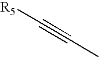
      wherein R₅ is as defined below,
   (v) a radical: 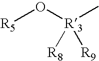
      wherein R′₃, R₅, R₈ and R₉ are as defined below,
   R₃ is a linear alkylene radical having from 1 to 6 carbon atoms, preferably —CH₂— or —(CH₂)₂—;
   R₂ is an alkyl radical having from 1 to 12 carbon atoms or an aryl, heteroaryl or aralkyl radical,
   R′₃, which is a divalent radical, is an alkylene radical having from 1 to 12 carbon atoms or an aryl, heteroaryl or aralkyl radical,
   R₄ is an alkyl radical having from 1 to 12 carbon atoms, an aryl, aralkyl or heteroaryl radical or a radical —COR₆,
   R₆ is as defined below,
   R₅, R₆ and R₇, which may be identical or different, are each a hydrogen atom, an alkyl radical having from 1 to 12 carbon atoms or an aryl, aralkyl or heteroaryl radical,
   R₈ and R₉, which may be identical or different, are each a hydrogen atom or a methyl radical,
   Ar is an aryl, heteroaryl or aralkyl radical,
   X is two hydrogen atoms, an oxygen atom or a sulfur atom,
   Y is an oxygen or sulfur atom,
   n is 0 or 1,
      and the optical and geometrical isomers of the said compounds of formula (I), and also the salts thereof.

### DETAILED DESCRIPTION OF BEST MODE AND SPECIFIC/PREFERRED EMBODIMENTS OF THE INVENTION

In particular, when the compounds according to the invention are in the form of salts, they are salts of an alkali metal or alkaline-earth metal, zinc salts or salts of an organic amine.

According to the present invention, the term “alkyl radical” means a linear or cyclic, optionally branched radical having from 1 to 12 carbon atoms, which may be interrupted with a hetero atom, and preferably the alkyl radicals having from 1 to 12 carbon atoms are methyl, ethyl, isopropyl, butyl, tert-butyl, hexyl, octyl, decyl or cyclohexyl radicals.

The term “aryl radical” means a phenyl, biphenyl, cinnamyl, indanyl or naphthyl radical, which may be mono- or polysubstituted, preferably disubstituted, with a halogen atom, a CF₃ radical, an alkyl radical having from 1 to 12 carbon atoms, an alkoxy radical having from 1 to 7 carbon atoms, a nitro function, a polyether radical, an aryl radical, a benzoyl radical, an alkyl ester group, a carboxylic acid, a hydroxyl radical optionally protected with an acetyl or benzoyl group or an amino function optionally protected with an acetyl or benzoyl group or optionally substituted with at least one alkyl radical having from 1 to 12 carbon atoms.

The term “aralkyl radical” means a benzyl, phenethyl or naphthalen-2-ylmethyl radical whose aromatic portion may be mono- or polysubstituted, preferably disubstituted, with a halogen atom, a CF₃ radical, an alkyl radical having from 1 to 12 carbon atoms, an alkoxy radical having from 1 to 7 carbon atoms, a nitro function, a polyether radical, an aryl radical, a benzoyl radical, an alkyl ester group, a carboxylic acid, a hydroxyl radical optionally protected with an acetyl or benzoyl group or an amino function optionally protected with an acetyl or benzoyl group or optionally substituted with at least one alkyl radical having from 1 to 12 carbon atoms.

The term “heteroaryl radical” means a radical selected from the group consisting of 4, 5, 6 or 7 membered rings containing 1, 2 or 3 heteroatoms such as N, S or O, such as the pyridyl, furyl, thienyl, isoxazolyl, oxadiazolyl, oxazolyl, isothiazolyl, quinazolinyl, benzothiadiazolyl, benzimidazolyl, indolyl, benzofuryl, pyrazolinyl or indolizinyl radical optionally substituted with at least one halogen, an alkyl radical having from 1 to 12 carbon atoms, an alkoxy radical having from 1 to 7 carbon atoms, an aryl radical, a nitro function, a polyether radical, a heteroaryl radical, a benzoyl radical, an alkyl ester group, a carboxylic acid, a hydroxyl radical optionally protected with an acetyl or benzoyl group or an amino function optionally protected with an acetyl or benzoyl group or optionally substituted with at least one alkyl radical having from 1 to 12 carbon atoms.

Among the compounds of formula (I) according to the present invention, mention may be made especially of the following compounds (alone or as a mixture):
1. 1-[1-(4-cyclohexylbenzoyl)-4-phenylpiperidin-4-yl]ethanone,
2. 1-(4-acetyl-4-phenylpiperidin-1-yl)-4-phenylbutan-1-one.
3. 1-{1-[5-(2-methyl-5-trifluoromethyl-2H-pyrazol-3-yl)thiophene-2-carbonyl]-4-phenylpiperidin-4-yl}ethanone,
4. 1-(4-acetyl-4-phenylpiperidin-1-yl)-3-(1-ethyl-3-methyl-1H-pyrazol-4-yl)propenone,
5. 1-{1-[5-(2-methyl-5-trifluoromethyl-2H-pyrazol-3-yl)thiophene-2-carbonyl]-4-phenylpiperidin-4-yl}butan-1-one,
6. 1-{4-phenyl-1-[3-pyridin-4-yl-1-(3-trifluoromethylphenyl)-1H-pyrazole-4-carbonyl]piperidin-4-yl}butan-1-one,
7. 1-{1-[2-(4-chlorophenyl)indolizine-1-carbonyl]-4-phenylpiperidin-4-yl}ethanone,
8. N-[2-(4-acetyl-4-phenylpiperidin-1-yl)-2-oxoethyl]-3-dimethylamino-N-methylbenzamide,
9. N-[2-(4-acetyl-4-phenylpiperidin-1-yl)-2-oxoethyl]-4-isopropyl-N-methylbenzamide,
10. N-[2-(4-acetyl-4-phenylpiperidin-1-yl)-2-oxoethyl]-4-dimethylaminobenzamide,
11. N-[3-(4-acetyl-4-phenylpiperidin-1-yl)-3-oxopropyl]-4-isobutylbenzamide,
12. 1,1-dimethyl-N-[3-(4-acetyl-4-phenylpiperidin-1-yl)-3-oxopropyl]indan-4-carboxamide,
13. 1,1-dimethyl-N-[2-(4-acetyl-4-phenylpiperidin-1-yl)-2-oxoethyl]indan-4-carboxamide,
14. 4-tert-butyl-N-[3-(4-butyryl-4-phenylpiperidin-1-yl)-3-oxopropyl]benzamide,
15. 1-(4-acetyl-4-phenylpiperidin-1-yl)-3-(1-ethyl-3,5-dimethyl-1H-pyrazol-4-yl)propenone,
16. N-[3-(4-acetyl-4-phenylpiperidin-1-yl)-3-oxopropyl]-4-cyclohexylbenzamide,
17. N-[2-(4-acetyl-4-phenylpiperidin-1-yl)-2-oxoethyl]-4-tert-butylbenzamide,
18. N-[2-(4-acetyl-4-phenylpiperidin-1-yl)-2-oxoethyl]-4-cyclohexylbenzamide,
19. 1-{1-[2-(4-chlorophenoxy)-2-methylpropionyl]-4-phenylpiperidin-4-yl}butan-1-one,
20. N-[2-(4-acetyl-4-phenylpiperidin-1-yl)-2-oxoethyl]-2-methanesulfonyl-N-methylbenzamide,
21. N-[2-(4-acetyl-4-phenylpiperidin-1-yl)-2-oxoethyl]4-butyl-N-methylbenzamide,
22. 1-{4-phenyl-1-[3-pyridin-4-yl-1-(3-trifluoromethylphenyl)-1H-pyrazole-4-carbonyl]piperidin-4-yl}ethanone,
23. N-[2-(4-acetyl-4-phenylpiperidin-1-yl)-2-oxoethyl]-N-methyl-N-biphenyl-4-carboxamide,
24. 1-{1-[5-(3-chlorophenoxy)-1,3-dimethyl-1 H-pyrazole-4-carbonyl]4-phenylpiperidin-4-yl}butan-1-one,
25. N-[2-(4-acetyl-4-phenylpiperidin-1-yl)-2-oxoethyl]-4-isobutyl-N-methylbenzamide,
26. 1,1-dimethyl-N-[2-(4-acetyl-4-phenylpiperidin-1-yl)-2-oxoethyl]indan-4-carboxamide,
27. 1-(4-acetyl-4-phenylpiperidin-1-yl)-2-(2-chlorophenoxy)-2-methylpropan-1-one,
28. 1-(4-acetyl-4-phenylpiperidin-1-yl)-3-(1-tert-butyl-3,5-dimethyl-1 H-pyrazol-4-yl)propenone,
29. N-[2-(4-butyryl-4-phenylpiperidin-1-yl)-2-oxoethyl]-3-dimethylamino-N-methylbenzamide,
30. N-[2-(4-acetyl-4-phenylpiperidin-1-yl)-2-oxoethyl]-4-isobutylbenzamide,
31. N-[2-(4-butyryl-4-phenylpiperidin-1-yl)-2-oxoethyl]-3-trifluoromethylbenzamide,
32. N-[2-(4-butyryl-4-phenylpiperidin-1-yl)-2-oxoethyl]-N-methyl-3-trifluoromethylbenzamide,
33. 1-(4-acetyl-4-phenylpiperidin-1-yl)-2-(4-chlorophenoxy)-2-methylpropan-1-one,
34. 1-(4-acetyl-4-phenylpiperidin-1-yl)-2-benzenesulfonylethanone,
35. 1-[1-(2-benzenesulfonylacetyl)-4-phenylpiperidin-4-yl]butan-1-one,
36. N-[2-(4-butyryl-4-phenylpiperidin-1-yl)-2-oxoethyl]biphenyl-4-carboxamide,
37. N-[2-(4-acetyl-4-phenylpiperidin-1-yl)-2-oxoethyl]-3-dimethylaminobenzamide,
38. 1-{1-[3-(1-tert-butyl-3,5-dimethyl-1 H-pyrazol-4-yl)acryloyl]-4-phenylpiperidin-4-yl}butan-1-one,
39. 1-{i-[2-(2-tert-butyl-6-methylphenoxy)acetyl]-4-phenylpiperidin-4-yl}butan-1-one,
40. 4-butyl-N-[2-(4-butyryl-4-phenylpiperidin-1-yl)-2-oxoethyl]benzamide,
41. N-[2-(4-acetyl-4-phenylpiperidin-1-yl)-2-oxoethyl]-4-butylbenzamide,
42. N-[2-(4-acetyl-4-phenylpiperidin-1-yl)-2-oxoethyl]4-isopropylbenzamide,
43. N-[3-(4-butyryl-4-phenylpiperidin-1-yl)-3-oxopropyl]4-cyclohexylbenzamide,
44. 1,1-dimethyl-N-[2-(4-butyryl-4-phenylpiperidin-1-yl)-2-oxoethyl]-N-methylindan-4-carboxamide,
45. N-[2-(4-butyryl-4-phenylpiperidin-1-yl)-2-oxoethyl]-2-methanesulfonyl-N-methylbenzamide,
46. 1-(1-{3-[3-(2,6-dichlorophenyl)-5-methylisoxazol-4-yl]acryloyl}-4-phenylpiperidin-4-yl)butan-1-one,
47. 1-{1-[2-(2-chlorophenoxy)-2-methylpropionyl]4-phenylpiperidin-4-yl}butan-1-one,
48. N-[3-(4-butyryl-4-phenylpiperidin-1-yl)-3-oxopropyl]biphenyl-4-carboxamide,
49. 3-(2-chlorophenyl)-5-methyl-N-[2-(4-acetyl-4-phenylpiperidin-1-yl)-2-oxoethyl]isoxazole-4-carboxamide,
50. 1-{1-[5-(3-chlorophenoxy)-1,3-dimethyl-1H-pyrazole-4-carbonyl]4-phenylpiperidin-4-yl}ethanone,
51. N-[2-(4-butyryl-4-phenylpiperidin-1-yl)-2-oxoethyl]-4-isobutyl-N-methylbenzamide,
52. N-[2-(4-butyryl-4-phenylpiperidin-1-yl)-2-oxoethyl]-4-isopropyl-N-methylbenzamide,
53. 1-{1-[3-(1-ethyl-3-methyl-1H-pyrazol-4-yl)acryloyl]-4-phenylpiperidin-4-yl}butan-1-one,
54. N-[2-(4-butyryl-4-phenylpiperidin-1-yl)-2-oxoethyl]-4-cyclohexylbenzamide,
55. N-[2-(4-butyryl-4-phenylpiperidin-1-yl)-2-oxoethyl]-N-methylnaphthalene-2-carboxamide,
56. N-[2-(4-acetyl-4-phenylpiperidin-1-yl)-2-oxoethyl]naphthalene-2-carboxamide,
57. N-[3-(4-butyryl-4-phenylpiperidin-1-yl)-3-oxopropyl]4-isobutylbenzamide,
58. 4-tert-butyl-N-[2-(4-butyryl-4-phenylpiperidin-1-yl)-2-oxoethyl]benzamide,
59. 1-(4-acetyl-4-phenylpiperidin-1-yl)-3-[3-(2,6-dichlorophenyl)-5-methylisoxazol-4-yl]propenone,
60. N-[2-(4-butyryl-4-phenylpiperidin-1-yl)-2-oxoethyl]-N-methylbiphenyl-4-carboxamide,
61. N-[2-(4-acetyl-4-phenylpiperidin-1-yl)-2-oxoethyl]-N-methylnaphthalene-2-carboxamide,
62. 1-(4-acetyl-4-phenylpiperidin-1-yl)-2-phenoxyethanone,
63. N-[2-(4-acetyl-4-phenylpiperidin-1-yl)-2-oxoethyl]biphenyl-4-carboxamide,
64. 1,1-dimethyl-N-[3-(4-butyryl-4-phenylpiperidin-1-yl)-3-oxopropyl]indan-4-carboxamide,
65. N-[2-(4-butyryl-4-phenylpiperidin-1-yl)-2-oxoethyl]-N-methyl-4-trifluoromethylbenzamide,
66. N-[2-(4-butyryl-4-phenylpiperidin-1-yl)-2-oxoethyl]4-isopropylbenzamide,
67. N-[3-(4-acetyl-4-phenylpiperidin-1-yl)-3-oxopropyl]-4-tert-butylbenzamide,
68. 1-{1-[3-(1-ethyl-3,5-dimethyl-1H-pyrazol-4-yl)acryloyl]-4-phenylpiperidin-4-yl}butan-1-one,
69. 1-(4-acetyl-4-phenylpiperidin-1-yl)-2-(2-tert-butyl-6-methylphenoxy)ethanone,
70. 3-(2-chlorophenyl)-5-methylisoxazole-4-carboxylic acid [2-(4-butyryl-4-phenylpiperidin-1-yl)-2-oxoethyl]amide,
71. 1-[1-(2-phenoxyacetyl)₄-phenylpiperidin-4-yl]butan-1-one,
72. 4-butyl-N-[3-(4-butyryl-4-phenylpiperidin-1-yl)-3-oxopropyl]benzamide,
73. N-[3-(4-acetyl-4-phenylpiperidin-1-yl)-3-oxopropyl]-4-isopropylbenzamide,
74. 1-(4-acetyl-4-phenylpiperidin-1-yl)-2-phenoxybutan-1-one,
75. N-[3-(4-butyryl-4-phenylpiperidin-1-yl)-3-oxpropyl]-4-trifluoromethylbenzamide,
76. N-[3-(4-butyryl-4-phenylpiperidin-1-yl)-3-oxopropyl]4-isopropylbenzamide,
77. N-[3-(4-butyryl-4-phenylpiperidin-1-yl)-3-oxopropyl]-3-trifluoromethylbenzamide,
78. N-[2-(4-butyryl-4-phenylpiperidin-1-yl)-2-oxoethyl]-2-methanesulfonylbenzamide,
79. N-[2-(4-butyryl-4-phenylpiperidin-1-yl)-2-oxoethyl]naphthalene-2-carboxamide,
80. 1-(4-butyryl-4-phenylpiperidin-1-yl)-2-phenoxybutan-1-one,
81. N-[3-(4-butyryl-4-phenylpiperidin-1-yl)-3-oxopropyl]-3-dimethylaminobenzamide,
82. N-[2-(4-butyryl-4-phenylpiperidin-1-yl)-2-oxoethyl]4-isobutylbenzamide,
83. N-[2-(4-butyryl-4-phenylpiperidin-1-yl)-2-oxoethyl]-3-dimethylaminobenzamide,
84. N-[3-(4-butyryl-4-phenylpiperidin-1-yl)-3-oxopropyl]4-dimethylaminobenzamide,
85. N-[2-(4-butyryl-4-phenylpiperidin-1-yl)-2-oxoethyl]-4-dimethylaminobenzamide,
86. biphenyl-4-carboxylic acid [3-(4-acetyl-4-phenylpiperidin-1-yl)-3-oxopropyl]amide,
87. N-[3-(4-butyryl-4-phenylpiperidin-1-yl)-3-oxopropyl]naphthalene-2-carboxamide,
88. N-[3-(4-butyryl-4-phenylpiperidin-1-yl)-3-oxopropyl]-2-methanesulfonylbenzamide,
89. N-[3-(4-acetyl-4-phenylpiperidin-1-yl)-3-oxopropyl]naphthalene-2-carboxamide.

According to the present invention, the compounds of formula (I) that are more particularly preferred are those that satisfy at least one of the following characteristics:
Ar is a phenyl radical,
Y is an oxygen atom,
R₂ is an alkyl radical, and preferably a methyl or propyl radical,
X is an oxygen atom,
R1 is the radical 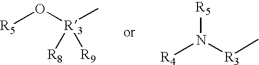
R₃ is an alkylene radical and preferably a methylene radical,
R₅ is an aryl radical [2-chlorophenyl] when R₁ is 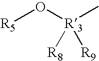
   and R₅ is a hydrogen atom when R₁ is the radical 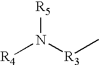
R₄ is 4-cyclohexylbenzoyl.

The invention also relates to the method for preparing the compounds of formula (I), as follows.

The ketopiperidine is coupled to a benzoic acid using coupling agents commonly employed in peptide synthesis, for instance HOBT/HBTU or HATU, optionally in the presence of a base such as triethylamine, in a solvent such as DMF or a mixture of solvents, for instance dichloromethane/DMF. The work-up is a series of extractions with an organic solvent and washing with water. If the coupled acid contains a protected amine function, this amine may be deprotected and then coupled in turn with another carboxylic acid according to the same coupling methods as previously.

The compounds according to the invention mentioned above were all obtained according to the preparation method described above and/or according to the synthetic methods known to those skilled in the art.

The compounds according to the invention have modulatory properties on the LXRβ-type receptors. The term “LXRβ receptors” generally means the LXRβ receptors taken individually and/or in the form of homodimers and/or in the form of heterodimers such as, without limitation, the LXR/RAR; LXR/LXR; LXR/PPAR; LXRNDR heterodimers, irrespective of the types used for each of the receptors mentioned.

This activity on the LXRβ receptors is measured in the transactivation test and quantified by means of the dissociation constant Kdapp (apparent), as described in Example 3.

The preferred compounds of the present invention have a dissociation constant of less than or equal to 10,000 nM and preferably less than or equal to 3,000 nM.

The present invention also features, as medicinal products, the compounds of formula (I) as described above.

The present invention also features formulating the compounds of formula (I) into pharmaceutical or cosmetic compositions more particularly useful for treating the following disorders, afflictions or complaints:
dermatological conditions or afflictions associated with a keratinization disorder relating to differentiation and proliferation, especially common acne, comedones, polymorphs, rosacea, nodulocystic acne, acne conglobata, senile acne and secondary acne such as solar, medicinal or occupational acne,
ichthyosis, ichthyosiform conditions, Darier's disease, palmoplantar keratoderma, leukoplakia and leukoplakiform conditions, and cutaneous or mucous (oral) lichen,
dermatological conditions or afflictions with an inflammatory immunoallergic component, with or without a cellular proliferation disorder, especially cutaneous, mucous or ungual psoriasis, psoriatic rheumatism, cutaneous atopy, such as eczema, respiratory atopy or gingival hypertrophy,
benign or malignant dermal or epidermal proliferations, whether or not of viral origin, especially common warts, flat warts, epidermodysplasia verruciformis, oral or florid papillomatoses and T lymphoma,
proliferations that may be induced by ultraviolet light, especially basocellular and spinocellular epithelioma,
precancerous skin lesions, especially keratoacanthomas,
immune dermatitides, especially lupus erythematosus,
bullous immune diseases,
collagen diseases, especially scleroderma,
dermatological or systemic conditions or afflictions with an immunological component,
skin disorders due to exposure to UV radiation, photo-induced or chronological aging of the skin or actinic pigmentations and keratoses, or any pathology associated with chronological or actinic aging, especially xerosis,
sebaceous function disorders, especially the hyperseborrhoea of acne, simple seborrhoea or seborrhoeic dermatitis,
cicatrization disorders or stretch marks,
pigmentation disorders, such as hyperpigmentation, melasma, hypopigmentation or vitiligo,
lipid metabolism conditions or afflictions, such as obesity, hyperlipidaemia, non-insulin-dependent diabetes or syndrome X,
inflammatory conditions such as arthritis,
cancerous or precancerous conditions,
alopecia of various origins, especially alopecia caused by chemotherapy or radiation,
immune system disorders, such as asthma, type I sugar diabetes, multiple sclerosis or other selective dysfunctions of the immune system, or
disorders of the cardiovascular system, such as arteriosclerosis or hypertension.

This invention also features pharmaceutical or cosmetic compositions comprising, formulated into a physiologically acceptable medium, at least one compound of formula (I) as defined above.

The compositions according to the invention may be administered enterally, parenterally, topically or ocularly. The pharmaceutical compositions are preferably packaged in a form which is suitable for topical application.

Via the enteral route, the composition, more particularly the pharmaceutical composition, may be in the form of tablets, gel capsules, sugar-coated tablets, syrups, suspensions, solutions, powders, granules, emulsions or lipid or polymer vesicles or nanospheres or microspheres to allow controlled release. Via the parenteral route, the composition may be in the form of solutions or suspensions for infusion or for injection.

The compounds according to the invention are generally administered at a daily dose of about 0.001 mg/kg to 100 mg/kg of body weight in 1 to 3 dosage intakes.

The compounds are administered systemically at a concentration generally of from 0.001% to 10% by weight and preferably from 0.01% to 1% by weight relative to the weight of the composition.

Via the topical route, the pharmaceutical composition according to the invention is more particularly useful for treating the skin and mucous membranes and may be in the form of ointments, creams, milks, salves, powders, impregnated pads, syndets, solutions, gels, sprays, foams, suspensions, stick lotions, shampoos or washing bases. It may also be in the form of suspensions of lipid or polymer vesicles or nanospheres or microspheres or polymer patches and hydrogels to allow controlled release. This topical-route composition may be in anhydrous form, in aqueous form or in the form of an emulsion.

The compounds are administered topically at a concentration generally of from 0.001% to 10% by weight, preferably from 0.01% to 1% by weight relative to the total weight of the composition.

The compounds according to the invention also find application in the cosmetic field, in particular in body and hair hygiene and especially for treating acne-prone skin, for combating the greasy appearance of the skin and the hair, in protecting against the harmful effects of sunlight or in treating physiologically dry skin, and for preventing and/or combating photo-induced and/or chronological aging.

This invention therefore also features the cosmetic use of a composition comprising, in a physiologically acceptable support, at least one of the compounds of formula (I) for body or hair hygiene.

The cosmetic compositions according to the invention having, in a cosmetically acceptable support, at least one compound of formula (I) or an optical or geometrical isomer thereof or a salt thereof, may usually be in the form of a cream, a milk, a lotion, a gel, suspensions of lipid or polymer vesicles or nanospheres or microspheres, impregnated pads, solutions, sprays, foams, sticks, soaps, shampoos or washing bases.

The concentration of compound of formula (I) in the cosmetic composition is from 0.001% to 3% by weight relative to the total weight of the composition.

The pharmaceutical and cosmetic compositions as described above may also contain inert or even pharmacodynamically active additives as regards the pharmaceutical compositions, or combinations of these additives, and especially:
wetting agents;
flavor enhancers;
preservatives such as para-hydroxybenzoic acid esters;
stabilizers;
humidity regulators;
pH regulators;
osmotic pressure modifiers;
emulsifiers;
UV-A and UV-B screening agents;
antioxidants, such as a-tocopherol, butylhydroxyanisole or butylhydroxytoluene, superoxide dismutase, ubiquinol or certain metal-chelating agents;
depigmenting agents such as hydroquinone, azelaic acid, caffeic acid or kojic acid;
emollients;
moisturizers, for instance glycerol, PEG 400, thiamorpholinone and derivatives thereof, or urea;
antiseborrhoeic or antiacne agents, such as S-carboxymethylcysteine, S-benzylcysteamine, salts thereof or derivatives thereof, or benzoyl peroxide; antibiotics, for instance erythromycin and its esters, neomycin, clindamycin and its esters, and tetracyclines;
antifungal agents such as ketoconazole or polymethylene-4,5-isothiazolidones-3;
agents for promoting regrowth of the hair, for instance Minoxidil (2,4-diamino-6-piperidinopyrimidine 3-oxide) and its derivatives, Diazoxide (7-chloro-3-methyl-1,2,4-benzothiadiazine 1,1-dioxide) and Phenyloin (5,4-diphenylimidazolidine-2,4-dione);
non-steroidal anti-inflammatory agents;
carotenoids, and especially β-carotene;
antipsoriatic agents such as anthraline and its derivatives;
eicosa-5,8,11,14-tetraynoic acid and eicosa-5,8,11-triynoic acid, and esters and amides thereof;
retinoids, i.e. RAR or RXR receptor ligands, which may be natural or synthetic;
corticosteroids or oestrogens;
α-hydroxy acids and α-keto acids or derivatives thereof, such as lactic acid, malic acid, citric acid, glycolic acid, mandelic acid, tartaric acid, glyceric acid or ascorbic acid, and also the salts, amides or esters thereof, or β-hydroxy acids or derivatives thereof, such as salicylic acid and the salts, amides or esters thereof;
ion-channel blockers such as potassium-channel blockers;
or alternatively, more particularly for the pharmaceutical compositions, in combination with medicinal products known to interfere with the immune system (for example cyclosporin, FK 506, glucocorticoids, monoclonal antibodies, cytokines or growth factors, etc.).

Of course, one skilled in this art will take care to select the optional compound(s) to be added to these compositions such that the advantageous properties intrinsically associated with the present invention are not, or are not substantially, adversely affected by the envisaged addition.

In order to further illustrate the present invention and the advantages thereof, the following specific examples are given, including several examples of the production of active compounds of formula (I), and the biological activities and specific formulations thereof, it being understood that same are intended only as illustrative and in nowise limitative. In said examples to follow, all parts and percentages are given by weight, unless otherwise indicated.

### EXAMPLE 1

### Synthesis of 1-[1-(4-cyclohexylbenzoyl)-4-phenylpiperidin-4-yl]ethanone

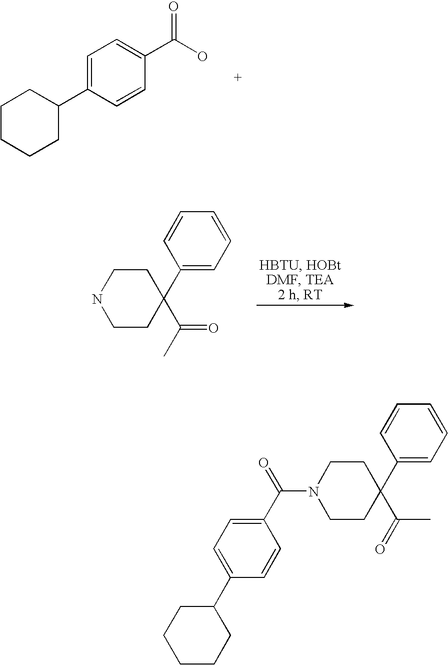

4-Cyclohexylbenzoic acid (204 mg, 1 mmol) in 6 ml of DMF is activated with a mixture of HOBT (135 mg, 1 mmol)/HBTU (379 mg, 1 mmol) in the presence of 3 equivalents of triethylamine (418 μl, 3 mmol) for 10 minutes at room temperature, followed by addition of 4-acetyl-4-phenylpiperidine hydrochloride (240 mg, 1 mmol). After 3 hours, the reaction medium is poured into 10 ml of ethyl acetate and washed with 0.1 M sodium bicarbonate solution and then with saturated sodium chloride solution. The organic phase is dried over magnesium sulfate, filtered and evaporated. The solid is taken up in a few millilitres of heptane, filtered off and dried to give 1-[1-(4-cyclohexylbenzoyl)₄-phenylpiperidin-4-yl]ethanone (340 mg, 87%). ¹H NMR (400 MHz, CDCl3): 1.23-1.26 (m, 5H), 1.73-1.85 (m, 6H), 1.94 (s, 3H), 2.2 (m, 1H), 2.35-2.51 (m, 3H), 3.35 (m, 2H), 3.3 (m, 1H), 4.3 (m, 1H), 7.21 (d, 2H), 7.30 (m, 5H), 7.38 (d, 2H).

### EXAMPLE 2

### Synthesis of 1-(4-acetyl-4-phenylpiperidin-1-yl)-4-phenylbutan-1-one

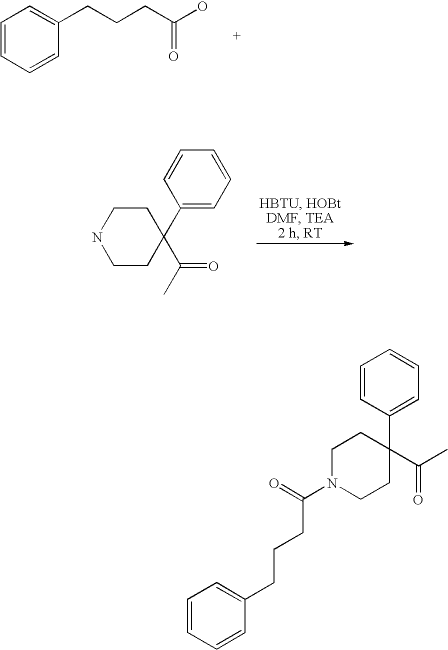

4-Phenylbutyric acid (164 m, 1 mmol) in 6 ml of DMF is activated with a mixture of HOBT (135 mg, 1 mmol)/HBTU (379 mg, 1 mmol) in the presence of 3 equivalents of triethylamine (418 μl, 3 mmol) for 10 minutes at room temperature, followed by addition of 4-acetyl-4-phenylpiperidine hydrochloride (240 mg, 1 mmol). After 2 hours, the reaction medium is poured into 10 ml of ethyl acetate and washed with 0.1M sodium bicarbonate solution and then with saturated sodium chloride solution. The organic phase is dried over magnesium sulfate, filtered and evaporated to give an oil, 1-(4-acetyl-4-phenylpiperidin-1-yl)-4-phenylbutan-1-one (326 mg, 93% crude).

### EXAMPLE 3

### LXRβ Activity, Agonists and Antagonists

The activity of the LXRβ receptors is measured in a transactivation test. Activation of the receptors with an agonist (activator) in HeLa cells leads to the expression of a reporter gene, luciferase, which, in the presence of a substrate, generates light. The activation of the receptors may thus be measured by quantifying the luminescence produced after incubating the cells in the presence of a reference agonist. The antagonist products displace the agonist from its site, thus preventing activation of the receptor: there will thus be a reduction in the light produced, which may be quantified. The agonist products are tested alone and their effect is measured by measuring the activation of luminescence after incubation.

Determination of the Kdapp:

In this study, a constant that is the affinity of the molecule for the receptor is determined. Since this value can fluctuate depending on the basal activity and the expression of the receptor, it is known as the Kd apparent (KdApp).

To determine this constant, “crossover curves” of the test product against a reference agonist are produced in a 96-well plate: 10 concentrations of the test product plus a concentration 0 (in the rows) and 7 concentrations of the agonist plus a 0 concentration (in the columns). This is 88 measurement points for one product and one receptor. The remaining 8 wells are used for the 100% control (total agonist) and 0% control (DMSO).

These crossover curves make it possible to determine the AC50 values (concentration at which 50% activation is observed) of the reference ligand at various concentrations of the test product. These AC50 values are used to calculate the Schild regression by plotting a straight line corresponding to the Schild equation (“quantitation in receptor pharmacology,” Terry P. Kenakin, Receptors and Channels, 2001, 7, 371-385).

In the case of an antagonist, an IC50 value (concentration inhibiting 50% of the activity) is calculated by plotting the curve of the product at the concentration of the reference ligand giving 80% activation.

The cell lines used are HG5LN cells, HeLa cells stably transfected with the (17mer)₅-bGlob-Luc reporter and also stably transported with the Gal-hLXRβ-DEF plasmid. These cells are inoculated into 96-well plates at a rate of 10 000 cells per well in 100 μl of DMEM medium free of phenol red and supplemented with 10% defatted calf serum. The plates are then incubated at 37° C. and 7% CO₂ for 4 hours.

The various dilutions of the test products, of the reference ligand and of the 100% control (N-(2,2,2-trifluoroethyl)-N-[4-(trifluorohydroxy-trifluoromethylethyl)phenyl]benzenesulfonamide) and of the 0% control (0.2% dimethyl sulfoxide) are added at a rate of 5 μl per well. The plates are then incubated for 18 hours at 37° C. and 7% CO₂.

The culture medium is removed by turning over and 100 μl of a 1:1 PBS/luciferine mixture are added to each well. After 5 minutes, the plates are read using a luminescence detector.

TABLE
Affinity for the LXRβ receptor: calculation of the Kd
App.:

| | | | KdR/ | |
|---|---|---|---|---|
| Test compound | KdR | KdA | KdA | KdApp |
| Compound 18: | 2,000 | 500 | 4 | 2,000 |
| N-[2-(4-Acetyl-4- | | | | |
| phenylpiperidin-1-yl)-2- | | | | |
| oxoethyl]-4- | | | | |
| cyclohexylbenzamide | | | | |
| Compound 47 | 2,000 | 20,000 | 0.1 | 2,000 |
| 1-{1-[2-(2-Chlorophenoxy)-2- | | | | |
| methylpropionyl]-4- | | | | |
| phenylpiperidin-4-yl}butan-1- | | | | |
| one | | | | |
| Compound 54: | 2.000 | 500 | 4 | 2,000 |
| N-[2-(4-Butyryl-4- | | | | |
| phenylpiperidin-1-yl)-2- | | | | |
| oxoethyl]-4- | | | | |
| cyclohexylbenzamide | | | | |

### EXAMPLE 4

This example illustrates various specific formulations based on the compounds according to the invention.

A-ORAL ROUTE:

(a) 0.2 g tablet:

| | | |
|---|---|---|
| Compound 18 | 0.001 | g |
| Starch | 0.114 | g |
| Dicalcium phosphate | 0.020 | g |
| Silica | 0.020 | g |
| Lactose | 0.030 | g |
| Talc | 0.010 | g |
| Magnesium stearate | 0.005 | g |

(b) Drinkable suspension in 5 ml ampoules:

| Compound 54 | 0.001 | g |
|---|---|---|
| Glycerol | 0.500 | g |
| 70% Sorbitol | 0.500 | g |
| Sodium saccharinate | 0.010 | g |
| Methyl para-hydroxybenzoate | 0.040 | g |
| Flavoring | qs | |
| Purified water | qs 5 | ml |

(c) 0.8 g tablet:

| | | |
|---|---|---|
| Compound of Example 1 | 0.500 | g |
| Pregelatinized starch | 0.100 | g |
| Microcrystalline cellulose | 0.115 | g |
| Lactose | 0.075 | g |
| Magnesium stearate | 0.010 | g |

(d) Drinkable suspension in 10 ml ampoules:

| Compound 20 | 0.200 | g |
|---|---|---|
| Glycerol | 1.000 | g |
| 70% Sorbitol | 1.000 | g |
| Sodium saccharinate | 0.010 | g |
| Methyl para-hydroxybenzoate | 0.080 | g |
| Flavoring | qs | |
| Purified water | qs 10 | ml |

B-TOPICAL ROUTE:

(a) Ointment:

| | | |
|---|---|---|
| Compound 54 | 0.020 | g |
| Isopropyl myristate | 81.700 | g |
| Liquid petroleum jelly fluid | 9.100 | g |
| Silica (“Aerosil 200” marketed by | 9.180 | g |
| Degussa) | | |

(b) Ointment:

| | | |
|---|---|---|
| Compound 47 | 0.300 | g |
| White petroleum jelly codex | qs 100 | g |

(c) Nonionic water-in-oil cream:

| | | |
|---|---|---|
| Compound 18 | 0.100 | g |
| Mixture of emulsifying lanolin alcohols, | 39.900 | g |
| waxes and oils (“Anhydrous Eucerin” | 0.075 | g |
| marketed by BDF) | | |
| Methyl para-hydroxybenzoate | 0.075 | g |
| Propyl para-hydroxybenzoate | 100 | g |

| | |
|---|---|
| Sterile demineralized water | qs |

(d) Lotion:

| | | |
|---|---|---|
| Compound 47 | 0.100 | g |
| Polyethylene glycol (PEG 400) | 69.900 | g |
| 95% Ethanol | 30.000 | g |

(e) Hydrophobic ointment:

| | | |
|---|---|---|
| Compound 54 | 0.300 | g |
| Isopropyl myristate | 36.400 | g |
| Silicone oil (“Rhodorsil 47 V 300” | 36.400 | g |
| marketed by Rhone-Poulenc) | | |
| Beeswax | 13.600 | g |
| Silicone oil (“Abil 300,000 cst” marketed | qs 100 | g |
| by Goldschmidt) | | |

(f) Nonionic oil-in-water cream:

| | | |
|---|---|---|
| Compound 18 | 1.000 | g |
| Cetyl alcohol | 4.000 | g |
| Glyceryl monostearate | 2.500 | g |
| PEG-50 stearate | 2.500 | g |
| Karite butter | 9.200 | g |
| Propylene glycol | 2.000 | g |
| Methyl para-hydroxybenzoate | 0.075 | g |
| Propyl para-hydroxybenzoate | 0.075 | g |
| Sterile demineralized water | qs 100 | g |

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A ligand compound having the following structural formula (I): 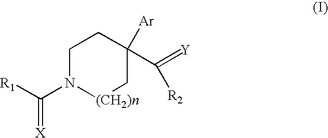
in which:
R₁ is:
(i) an alkyl, aryl, aralkyl, aralkenyl or heteroaryl radical,
(ii) a radical: 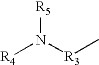
wherein R₃, R₄ and R₅ are as defined below,
(iii) a radical: 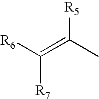
wherein R₆ and R₇ are as defined below,
(iv) a radical: 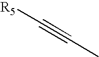
wherein R₅ is as defined below,
(v) a radical: 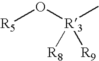
wherein R₁₃, R₅, R₈ and R₉ are as defined below,
R₃ is a linear alkylene radical having from 1 to 6 carbon atoms,
R₂ is an alkyl radical having from 1 to 12 carbon atoms or an aryl, heteroaryl or aralkyl radical,
R′₃, which is a divalent radical, is an alkylene radical having from 1 to 12 carbon atoms or an aryl, heteroaryl or aralkyl radical,
R₄ is an alkyl, aryl, aralkyl or heteraryl radical or a radical —COR₆, wherein R₆ is as defined below,
R₅, R₆ and R₇, which may be identical or different, are each a hydrogen atom or an alkyl, an aryl, aralkyl or heteroaryl radical,
R₈ and R₉, which may be identical or different, are each a hydrogen atom or a methyl radical,
Ar is an aryl, heteroaryl or aralkyl radical,
X is two hydrogen atoms, an oxygen atom or a sulfur atom,
Y is an oxygen or sulfur atom,
n is 0 or 1,
and the optical and geometrical isomers and salts of the said compounds of formula (I).

2. The ligand compound as defined by claim 1, wherein formula (I), R₁ is a radical (i).

3. The ligand compound as defined by claim 1, wherein formula (I), R₁ is a radical (ii).

4. The ligand compound as defined by claim 1, wherein formula (I), R₁ is a radical (iii).

5. The ligand compound as defined by claim 1, wherein formula (I), R₁ is a radical (iv).

6. The ligand compound as defined by claim 1, wherein formula (I), R₁ is a radical (v).

7. An alkali metal or alkaline-earth metal, or zinc or organic amine salt of the ligand compound as defined by claim 1.

8. The ligand compound as defined by claim 1, comprising at least one alkyl radical substituted selected from the group consisting of linear or cyclic, optionally branched radicals having from 1 to 12 carbon atoms, which may be interrupted with a hetero atom.

9. The ligand compound as defined by claim 8, comprising at least one methyl, ethyl, isopropyl, butyl, tert-butyl, hexyl, octyl, decyl or cyclohexyl radical substituent.

10. The ligand compound as defined by claim 1, comprising at least one phenyl, biphenyl, cinnamyl, indanyl or naphthyl radical substituent, which may be mono- or disubstituted with a halogen atom, a CF₃ radical, an alkyl radical having from 1 to 12 carbon atoms, an alkoxy radical having from 1 to 7 carbon atoms, a nitro function, a polyether radical, an aryl radical, a benzoyl radical, an alkyl ester group, a carboxylic acid, a hydroxyl radical optionally protected with an acetyl or benzoyl group or an amino function optionally protected with an acetyl or benzoyl group or optionally substituted with at least one alkyl radical having from 1 to 12 carbon atoms.

11. The ligand compound as defined by claim 1, comprising at least one benzyl, phenethyl or naphthalen-2-ylmethyl radical substituent, the aromatic moiety of which may be mono- or disubstituted with a halogen atom, a CF₃ radical, an alkyl radical having from 1 to 12 carbon atoms, an alkoxy radical having from 1 to 7 carbon atoms, a nitro function, a polyether radical, an aryl radical, a benzoyl radical, an alkyl ester group, a carboxylic acid, a hydroxyl radical optionally protected with an acetyl or benzoyl group or an amino function optionally protected with an acetyl or benzoyl group or optionally substituted with at least one alkyl radical having from 1 to 12 carbon atoms.

12. The ligand compound as defined by claim 1, comprising at least one pyridyl, furyl, thienyl, isoxazolyl, oxadiazolyl, oxazolyl, benzimidazolyl, indolyl, benzofuryl, pyrazolinyl or indolizinyl radical substituent optionally substituted with at least one halogen, an alkyl radical having from 1 to 12 carbon atoms, an alkoxy radical having from 1 to 7 carbon atoms, an aryl radical, a nitro function, a polyether radical, a heteroaryl radical, a benzoyl radical, an alkyl ester group, a carboxylic acid, a hydroxyl radical optionally protected with an acetyl or benzoyl group or an amino function optionally protected with an acetyl or benzoyl group or optionally substituted with at least one alkyl radical having from 1 to 12 carbon atoms.

13. The ligand compound as defined by claim 1, selected from the group consisting of:
1. 1-[1-(4-cyclohexylbenzoyl)₄-phenylpiperidin-4-yl]ethanone,
2. 1-(4-acetyl-4-phenylpiperidin-1-yl)-4-phenylbutan-1-one,
3. 1-{1-[5-(2-methyl-5-trifluoromethyl-2H-pyrazol-3-yl)thiophene-2-carbonyl]-4-phenylpiperidin-4-yl}ethanone,
4. 1-(4-acetyl-4-phenylpiperidin-1-yl)-3-(1-ethyl-3-methyl-1H-pyrazol-4-yl)propenone,
5. 1-{1-[5-(2-methyl-5-trifluoromethyl-2H-pyrazol-3-yl)thiophene-2-carbonyl]4-phenylpiperidin-4-yl}butan-1-one,
6. 1-{4-phenyl-1-[3-pyridin-4-yl-1-(3-trifluoromethylphenyl)-1H-pyrazole-4-carbonyl]piperidin-4-yl}butan-1-one,
7. 1-{1-[2-(4-chlorophenyl)indolizine-1-carbonyl]-4-phenylpiperidin-4-yl}ethanone,
8. N-[2-(4-acetyl-4-phenylpiperidin-1-yl)-2-oxoethyl]-3-dimethylamino-N-methylbenzamide,
9. N-[2-(4-acetyl-4-phenylpiperidin-1-yl)-2-oxoethyl]-4-isopropyl-N-methylbenzamide,
10. N-[2-(4-acetyl-4-phenylpiperidin-1-yl)-2-oxoethyl]4-dimethylaminobenzamide,
11. N-[3-(4-acetyl-4-phenylpiperidin-1-yl)-3-oxopropyl]-4-isobutylbenzamide,
12. 1,1-dimethyl-N-[3-(4-acetyl-4-phenylpiperidin-1-yl)-3-oxopropyl]indan-4-carboxamide,
13. 1,1-dimethyl-N-[2-(4-acetyl-4-phenylpiperidin-1-yl)-2-oxoethyl]indan-4-carboxamide,
14. 4-tert-butyl-N-[3-(4-butyryl-4-phenylpiperidin-1-yl)-3-oxopropyl]benzamide,
15. 1-(4-acetyl-4-phenylpiperidin-1-yl)-3-(1-ethyl-3,5-dimethyl-1H-pyrazol-4-yl)propenone,
16. N-[3-(4-acetyl-4-phenylpiperidin-1-yl)-3-oxopropyl]-4-cyclohexylbenzamide,
17. N-[2-(4-acetyl-4-phenylpiperidin-1-yl)-2-oxoethyl]-4-tert-butylbenzamide,
18. N-[2-(4-acetyl-4-phenylpiperidin-1-yl)-2-oxoethyl]4-cyclohexylbenzamide,
19. 1-{1-[2-(4-chlorophenoxy)-2-methylpropionyl]4-phenylpiperidin-4-yl}butan-1-one,
20. N-[2-(4-acetyl-4-phenylpiperidin-1-yl)-2-oxoethyl]-2-methanesulfonyl-N-methylbenzamide,
21. N-[2-(4-acetyl-4-phenylpiperidin-1-yl)-2-oxoethyl]4-butyl-N-methylbenzamide,
22. 1-{4-phenyl-1-[3-pyridin-4-yl-1-(3-trifluoromethylphenyl)-1H-pyrazole-4-carbonyl]piperidin-4-yl}ethanone,
23. N-[2-(4-acetyl-4-phenylpiperidin-1-yl)-2-oxoethyl]-N-methyl-N-biphenyl-4-carboxamide,
24. 1-{1-[5-(3-chlorophenoxy)-1,3-dimethyl-1H-pyrazole-4-carbonyl]-4-phenylpiperidin-4-yl}butan-1-one,
25. N-[2-(4-acetyl-4-phenylpiperidin-1-yl)-2-oxoethyl]-4-isobutyl-N-methylbenzamide,
26. 1,1-dimethyl-N-[2-(4-acetyl-4-phenylpiperidin-1-yl)-2-oxoethyl]indan-4-carboxamide,
27. 1-(4-acetyl-4-phenylpiperidin-1-yl)-2-(2-chlorophenoxy)-2-methylpropan-1-one,
28. 1-(4-acetyl-4-phenylpiperidin-1-yl)-3-(1-tert-butyl-3,5-dimethyl-1H-pyrazol-4-yl)propenone,
29. N-[2-(4-butyryl-4-phenylpiperidin-1-yl)-2-oxoethyl]-3-dimethylamino-N-methylbenzamide,
30. N-[2-(4-acetyl-4-phenylpiperidin-1-yl)-2-oxoethyl]4-isobutylbenzamide,
31. N-[2-(4-butyryl-4-phenylpiperidin-1-yl)-2-oxoethyl]-3-trifluoromethylbenzamide,
32. N-[2-(4-butyryl-4-phenylpiperidin-1-yl)-2-oxoethyl]-N-methyl-3-trifluoromethylbenzamide,
33. 1-(4-acetyl-4-phenylpiperidin-1-yl)-2-(4-chlorophenoxy)-2-methylpropan-1-one,
34. 1-(4-acetyl-4-phenylpiperidin-1-yl)-2-benzenesulfonylethanone,
35. 1-[1-(2-benzenesulfonylacetyl)-4-phenylpiperidin-4-yl]butan-1-one,
36. N-[2-(4-butyryl-4-phenylpiperidin-1-yl)-2-oxoethyl]biphenyl-4-carboxamide,
37. N-[2-(4-acetyl-4-phenylpiperidin-1-yl)-2-oxoethyl]-3-dimethylaminobenzamide,
38. 1-{1-[3-(1-tert-butyl-3,5-dimethyl-1H-pyrazol-4-yl)acryloyl]-4-phenylpiperidin-4-yl}butan-1-one,
39. 1-{1-[2-(2-tert-butyl-6-methylphenoxy)acetyl]-4-phenylpiperidin-4-yl}butan-1-one,
40. 4-butyl-N-[2-(4-butyryl-4-phenylpiperidin-1-yl)-2-oxoethyl]benzamide,
41. N-[2-(4-acetyl-4-phenylpiperidin-1-yl)-2-oxoethyl]4-butylbenzamide,
42. N-[2-(4-acetyl-4-phenylpiperidin-1-yl)-2-oxoethyl]4-isopropylbenzamide,
43. N-[3-(4-butyryl-4-phenylpiperidin-1-yl)-3-oxopropyl]-4-cyclohexylbenzamide,
44. 1,1-dimethyl-N-[2-(4-butyryl-4-phenylpiperidin-1-yl)-2-oxoethyl]-N-methylindan-4-carboxamide,
45. N-[2-(4-butyryl-4-phenylpiperidin-1-yl)-2-oxoethyl]-2-methanesulfonyl-N-methylbenzamide,
46. 1-(1-{3-[3-(2,6-dichlorophenyl)-5-methylisoxazol-4-yl]acryloyl}-4-phenylpiperidin-4-yl)butan-1-one,
47. 1-{1-[2-(2-chlorophenoxy)-2-methylpropionyl]-4-phenylpiperidin-4-yl}butan-1-one,
48. N-[3-(4-butyryl-4-phenylpiperidin-1-yl)-3-oxopropyl]biphenyl-4-carboxamide,
49. 3-(2-chlorophenyl)-5-methyl-N-[2-(4-acetyl-4-phenylpiperidin-1-yl)-2-oxoethyl]isoxazole-4-carboxamide,
50. 1-{1-[5-(3-chlorophenoxy)-1,3-dimethyl-1H-pyrazole-4-carbonyl]-4-phenylpiperidin-4-yl}ethanone,
51. N-[2-(4-butyryl-4-phenylpiperidin-1-yl)-2-oxoethyl]4-isobutyl-N-methylbenzamide,
52. N-[2-(4-butyryl-4-phenylpiperidin-1-yl)-2-oxoethyl]-4-isopropyl-N-methylbenzamide,
53. 1-{1-[3-(1-ethyl-3-methyl-1H-pyrazol-4-yl)acryloyl]4-phenylpiperidin-4-yl}butan-1-one,
54. N-[2-(4-butyryl-4-phenylpiperidin-1-yl)-2-oxoethyl]4-cyclohexylbenzamide,
55. N-[2-(4-butyryl-4-phenylpiperidin-1-yl)-2-oxoethyl]-N-methylnaphthalene-2-carboxamide,
56. N-[2-(4-acetyl-4-phenylpiperidin-1-yl)-2-oxoethyl]naphthalene-2-carboxamide,
57. N-[3-(4-butyryl-4-phenylpiperidin-1-yl)-3-oxopropyl]-4-isobutylbenzamide,
58. 4-tert-butyl-N-[2-(4-butyryl-4-phenylpiperidin-1-yl)-2-oxoethyl]benzamide,
59. 1-(4-acetyl-4-phenylpiperidin-1-yl)-3-[3-(2,6-dichlorophenyl)-5-methylisoxazol-4-yl]propenone,
60. N-[2-(4-butyryl-4-phenylpiperidin-1-yl)-2-oxoethyl]-N-methylbiphenyl-4-carboxamide,
61. N-[2-(4-acetyl-4-phenylpiperidin-1-yl)-2-oxoethyl]-N-methylnaphthalene-2-carboxamide,
62. 1-(4-acetyl-4-phenylpiperidin-1-yl)-2-phenoxyethanone,
63. N-[2-(4-acetyl-4-phenylpiperidin-1-yl)-2-oxoethyl]biphenyl-4-carboxamide,
64. 1,1-dimethyl-N-[3-(4-butyryl-4-phenylpiperidin-1-yl)-3-oxopropyl]indan-4-carboxamide,
65. N-[2-(4-butyryl-4-phenylpiperidin-1-yl)-2-oxoethyl]-N-methyl-4-trifluoromethylbenzamide,
66. N-[2-(4-butyryl-4-phenylpiperidin-1-yl)-2-oxoethyl]-4-isopropylbenzamide,
67. N-[3-(4-acetyl-4-phenylpiperidin-1-yl)-3-oxopropyl]-4-tert-butylbenzamide,
68. 1-{1-[3-(1-ethyl-3,5-dimethyl-1 H-pyrazol-4-yl)acryloyl]-4-phenylpiperidin-4-yl}butan-1-one,
69. 1-(4-acetyl-4-phenylpiperidin-1-yl)-2-(2-tert-butyl-6-methylphenoxy)ethanone,
70. 3-(2-chlorophenyl)-5-methylisoxazole-4-carboxylic acid [2-(4-butyryl-4-phenylpiperidin-1-yl)-2-oxoethyl]amide,
71. 1-[1-(2-phenoxyacetyl)₄-phenylpiperidin-4-yl]butan-1-one,
72. 4-butyl-N-[3-(4-butyryl-4-phenylpiperidin-1-yl)-3-oxopropyl]benzamide,
73. N-[3-(4-acetyl-4-phenylpiperidin-1-yl)-3-oxopropyl]4-isopropylbenzamide,
74. 1-(4-acetyl-4-phenylpiperidin-1-yl)-2-phenoxybutan-1-one,
75. N-[3-(4-butyryl-4-phenylpiperidin-1-yl)-3-oxopropyl]-4-trifluoromethylbenzamide,
76. N-[3-(4-butyryl-4-phenylpiperidin-1-yl)-3-oxopropyl]-4-isopropylbenzamide,
77. N-[3-(4-butyryl-4-phenylpiperidin-1-yl)-3-oxopropyl]-3-trifluoromethylbenzamide,
78. N-[2-(4-butyryl-4-phenylpiperidin-1-yl)-2-oxoethyl]-2-methanesulfonylbenzamide,
79. N-[2-(4-butyryl-4-phenylpiperidin-1-yl)-2-oxoethyl]naphthalene-2-carboxamide,
80. 1-(4-butyryl-4-phenylpiperidin-1-yl)-2-phenoxybutan-1-one,
81. N-[3-(4-butyryl-4-phenylpiperidin-1-yl)-3-oxopropyl]-3-dimethylaminobenzamide,
82. N-[2-(4-butyryl-4-phenylpiperidin-1-yl)-2-oxoethyl]-4-isobutylbenzamide,
83. N-[2-(4-butyryl-4-phenylpiperidin-1-yl)-2-oxoethyl]-3-dimethylaminobenzamide,
84. N-[3-(4-butyryl-4-phenylpiperidin-1-yl)-3-oxopropyl]-4-dimethylaminobenzamide,
85. N-[2-(4-butyryl-4-phenylpiperidin-1-yl)-2-oxoethyl]-4-dimethylaminobenzamide,
86. biphenyl-4-carboxylic acid [3-(4-acetyl-4-phenylpiperidin-1-yl)-3-oxopropyl]amide,
87. N-[3-(4-butyryl-4-phenylpiperidin-1-yl)-3-oxopropyl]naphthalene-2-carboxamide,
88. N-[3-(4-butyryl-4-phenylpiperidin-1-yl)-3-oxopropyl]-2-methanesulfonylbenzamide,
89. N-[3-(4-acetyl-4-phenylpiperidin-1-yl)-3-oxopropyl]naphthalene-2-carboxamide, and mixtures thereof.

14. The ligand compound as defined by claim 1, wherein formula (I) at least one of the following conditions is satisfied:
Ar is a phenyl radical,
Y is an oxygen atom,
R₂ is an alkyl radical,
X is an oxygen atom,
R1 is the radical: 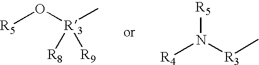
R₃ is an alkylene radical,
R₅ is an aryl radical when R₁ is 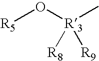
and R₅ is a hydrogen atom when R₁ is the radical 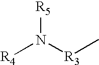
and R₄ is 4-cyclohexylbenzoyl.

15. A cosmetic/pharmaceutical composition useful for modulating LXR receptors, comprising a thus effective amount of at least one ligand compound as defined by claim 1, formulated into a physiologically acceptable medium therefor.

16. The cosmetic/pharmaceutical composition as defined by claim 15, comprising from 0.001% to 10% by weight of said at least one ligand compound.

17. The cosmetic/pharmaceutical composition as defined by claim 15, comprising from 0.01% to 1% by weight of said at least one ligand compound.

18. The cosmetic/pharmaceutical composition as defined by claim 15, formulated for topical application.

19. A regime or regimen for treating the following disorders, conditions or afflictions:
dermatological complaints associated with a keratinization disorder relating to differentiation and proliferation, common acne, comedones, polymorphs, rosacea, nodulocystic acne, acne conglobata, senile acne and secondary acne, or solar, medicinal or occupational acne,
ichthyosis, ichthyosiform conditions, Darier's disease, palmoplantar keratoderma, leukoplakia and leukoplakiform conditions, and cutaneous or mucous (oral) lichen,
dermatological complaints with an inflammatory immunoallergic component, with or without a cellular proliferation disorder, cutaneous, mucous or ungual psoriasis, psoriatic rheumatism, cutaneous atopy, eczema, respiratory atopy or gingival hypertrophy,
benign or malignant dermal or epidermal proliferations, whether or not of viral origin, common warts, flat warts, epidermodysplasia verruciformis, oral or florid papillomatoses and T lymphoma,
proliferations induced by ultraviolet light, basocellular and spinocellular epithelioma,
precancerous skin lesions, keratoacanthomas,
immune dermatitides, lupus erythematosus,
bullous immune diseases,
collagen diseases, scleroderma,
dermatological or systemic complaints with an immunological component,
skin disorders due to exposure to UV radiation, photo-induced or chronological aging of the skin or actinic pigmentations and keratoses, or any pathology associated with chronological or actinic aging, xerosis,
sebaceous function disorders, hyperseborrhoea of acne, simple seborrhoea or seborrhoeic dermatitis,
cicatrization disorders or stretch marks,
pigmentation disorders, hyperpigmentation, melasma, hypopigmentation or vitiligo,
lipid metabolism complaints, obesity, hyperlipidaemia, non-insulin-dependent diabetes or syndrome X,
inflammatory complaints, arthritis, cancerous or precancerous conditions,
alopecia of various origins, alopecia caused by chemotherapy or radiation,
immune system disorders, asthma, type I sugar diabetes, multiple sclerosis or other selective dysfunctions of the immune system, or
complaints of the cardiovascular system, arteriosclerosis or hypertension,
comprising administering to a mammalian organism in need of such treatment, a thus effective amount of at least one ligand compound as defined by claim 1.

20. A regime or regimen for treating an imbalance in the barrier function of the skin, comprising administering to a mammalian organism in need of such treatment, a thus effective amount of at least one ligand compound as defined by claim 1.

21. A regime or regimen for treating a disorder, condition or affliction associated with the LXR receptors, comprising administering to a mammalian organism in need of such treatment, a thus effective amount of at least one ligand compound as defined by claim 1.

22. A regime or regimen for treating a disorder, condition or affliction of differentiation and/or proliferation of epidermal cells, comprising administering to a mammalian organism in need of such treatment, a thus effective amount of at least one ligand compound as defined by claim 1.
